(19) United States
(12) Patent Application Publication
(10) Pub. No.: US 2025/0201374 A1
(43) Pub. Date: Jun. 19, 2025

(54) PHARMACY MANAGEMENT AND FULFILLMENT SYSTEMS AND METHODS

(71) Applicant: CP Hub LLC, Schaumburg, IL (US)

(72) Inventors: Brian Prigge, Barrington, IL (US); Kent McCullough, Mukwonago, WI (US); Pritesh Patel, Naperville, IL (US); Bhavesh Patel, Schaumburg, IL (US)

(21) Appl. No.: 18/981,011

(22) Filed: Dec. 13, 2024

Related U.S. Application Data
(60) Provisional application No. 63/610,908, filed on Dec. 15, 2023.

(51) Int. Cl. G16H 20/10; G16H 40/20

(52) U.S. Cl. CPC G16H 20/10; G16H 40/20

(57) ABSTRACT

Systems and methods for the processing and tracking of medication prescription audits are provided.

### CROSS-REFERENCE TO RELATED APPLICATION

This application claims the benefit of U.S. Provisional Patent Application No. 63/610,908 filed on Dec. 15, 2023, which is fully incorporated by reference in its entirety herein.

### TECHNICAL FIELD

This application relates to systems and methods for the management and fulfillment of medication prescriptions by pharmacies, including the automated processing of requests from pharmacy benefit managers for audits involving prescriptions that have been fulfilled by the pharmacies.

### BACKGROUND

Patients commonly utilize pharmacies to get prescriptions filled to obtain medications ordered by prescribers, such as doctors, dentists, and other medical professionals. The prescriptions typically include details regarding the patients, the prescribers, and the medications so that the pharmacies can dispense the medications accurately. For example, a prescription may include information identifying the patient, the prescriber, the name of the prescribed medication, the amount of the medication to dispense, and/or instructions for how the medication should be taken (e.g., dosages, frequency of taking the medication, etc.). Prescriptions may typically be received from prescribers by pharmacies on paper, electronically, and/or via fax.

The pharmacies may work with entities such as insurance companies and pharmacy benefit managers to determine patient benefits, such as whether the medications are covered, the total cost of the medications, co-pay amounts, etc. In general, the pharmacies may verify the prescriptions to ensure they are authorized and legitimate, then fill the prescriptions and dispense the prescribed medications to the patients. The pharmacies may also perform other tasks when filling prescriptions, such as looking for potential interactions with other medications being taken by the patients.

Pharmacy benefit managers routinely request audits from pharmacies that relate to the prescriptions that have been previously filled in order to determine whether the prescriptions were filled in compliance with applicable laws, regulations, and insurance company and pharmacy benefit manager requirements. For example, a prescription may have been filled with inaccurate directions, insufficient documentation, and/or be out of compliance with co-pay payment guidelines. An audit of such prescriptions may result in a clawback by the pharmacy benefit manager to request payment for the initial incorrect payments.

Audits may be burdensome and time consuming, due to the need for pharmacies to provide documentation to the pharmacy benefit managers for particular prescriptions. It may be especially difficult to timely respond to audit requests since pharmacies typically handle, process, and fill prescriptions for many types of medications and for many patients and prescribers at multiple locations. Moreover, pharmacy benefit managers may have different requirements regarding the documentation needed to respond to audit requests.

Therefore, there is a need for systems and methods for automating and tracking audits of medication prescriptions, in order to ensure that the audits are being timely processed.

### SUMMARY

The inventions described herein are intended to the solve the above-noted problems by providing systems and methods that are designed to, among other things: (1) provide prescription audit management to enable users, e.g., compliance managers at pharmacies, to accurately track and process audits; and (2) automate some or all of the processing of audit requests, e.g., received from pharmacy benefit managers, to assist with timely and accurately responding to the audit requests.

These and other embodiments, and various permutations and aspects, will become apparent and be more fully understood from the following detailed description and accompanying drawings, which set forth illustrative embodiments that are indicative of the various ways in which the principles of the invention may be employed.

### BRIEF DESCRIPTION OF THE DRAWINGS

FIG. 1 is a block diagram illustrating a system for processing and handling prescription audit requests between a pharmacy benefit manager and a pharmacy.

FIGS. 2A-2B are flowcharts illustrating operations for processing and handling prescription audit requests using the system of FIG. 1.

FIGS. 3-21 are exemplary screenshots of a user interface related to the system of FIG. 1 and the process of FIGS. 2A-2B.

### DETAILED DESCRIPTION

The description that follows describes, illustrates and exemplifies one or more particular embodiments of the invention in accordance with its principles. This description is not provided to limit the invention to the embodiments described herein, but rather to explain and teach the principles of the invention in such a way to enable one of ordinary skill in the art to understand these principles and, with that understanding, be able to apply them to practice not only the embodiments described herein, but also other embodiments that may come to mind in accordance with these principles. The scope of the invention is intended to cover all such embodiments that may fall within the scope of the appended claims, either literally or under the doctrine of equivalents.

It should be noted that in the description and drawings, like or substantially similar elements may be labeled with the same reference numerals. However, sometimes these elements may be labeled with differing numbers, such as, for example, in cases where such labeling facilitates a more clear description. Additionally, the drawings set forth herein are not necessarily drawn to scale, and in some instances proportions may have been exaggerated to more clearly depict certain features. Such labeling and drawing practices do not necessarily implicate an underlying substantive purpose. As stated above, the specification is intended to be taken as a whole and interpreted in accordance with the principles of the invention as taught herein and understood to one of ordinary skill in the art.

The systems and methods described herein can automate and track audits of medication prescriptions to improve a pharmacy's handling of the processing of the audits, including improving data capture of information related to the prescriptions and audits. The accurate and timely processing of prescription audits can help a pharmacy to maintain compliance with contractual requirements and the expectations of a pharmacy benefit manager, as well as to reduce risk exposure related to payment discrepancies. As a result, both pharmacy benefit managers that request audits and pharmacies that receive and process the audits may benefit from the use of the system and methods described herein.

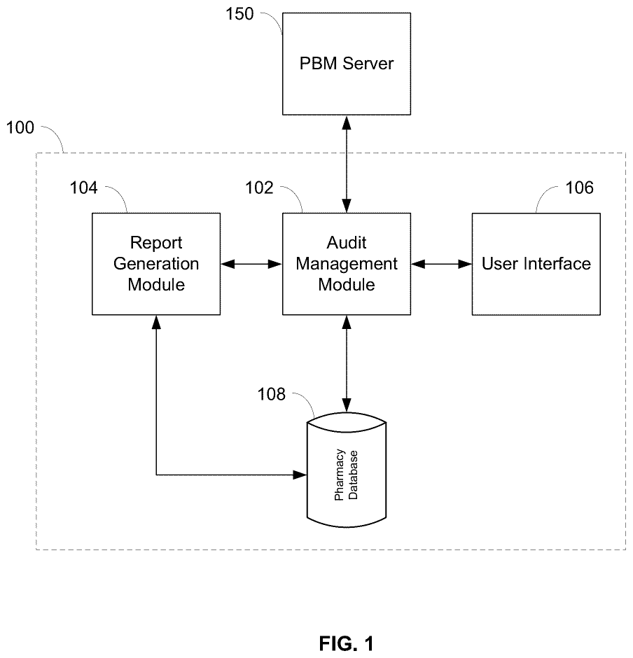

FIG. 1 illustrates a medication prescription audit system 100 for processing and handling prescription audit requests between a pharmacy benefit manager and a pharmacy. The system 100 may include modules and components that are connected through a network such as the Internet, which can facilitate communications through secure channels. The system 100 may be part of a pharmacy, and may be configured to receive and process prescription audit requests from a pharmacy benefit manager, e.g., from pharmacy benefit manager server 150, in order to improve the tracking and processing of the prescription audit requests. An audit management module 102 of the system 100 may communicate with the pharmacy benefit manager server 150, and also be in communication with a report generation module 104, a user interface 106, and a database 108.

In embodiments, some or all of the system 100 and/or software that implements the system 100 may be stored and/or be executable on a computing device. The computing device may be, for example, a personal computer (PC), a laptop, a tablet, a mobile device, a thin client, or other computing platform. The computing device may operate using a suitable operating system, such as Windows, Mac OS, iOS, and Android. In other embodiments, some or all of the system 100 and/or the software may be stored and/or executable on a remote server. In further embodiments, some or all of the system 100 and/or the software may be executable on standard web browsers, such as Chrome, Safari, Firefox, etc.

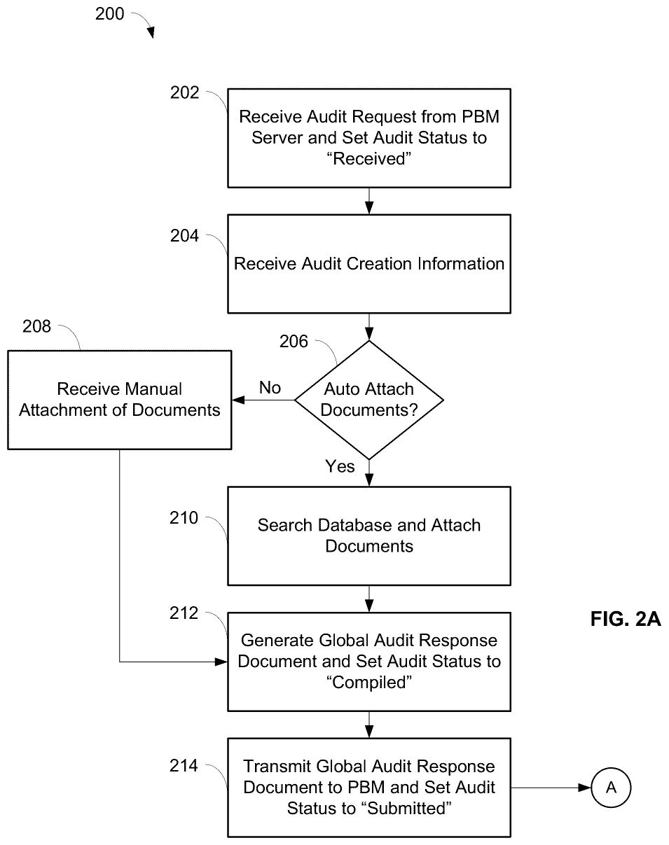

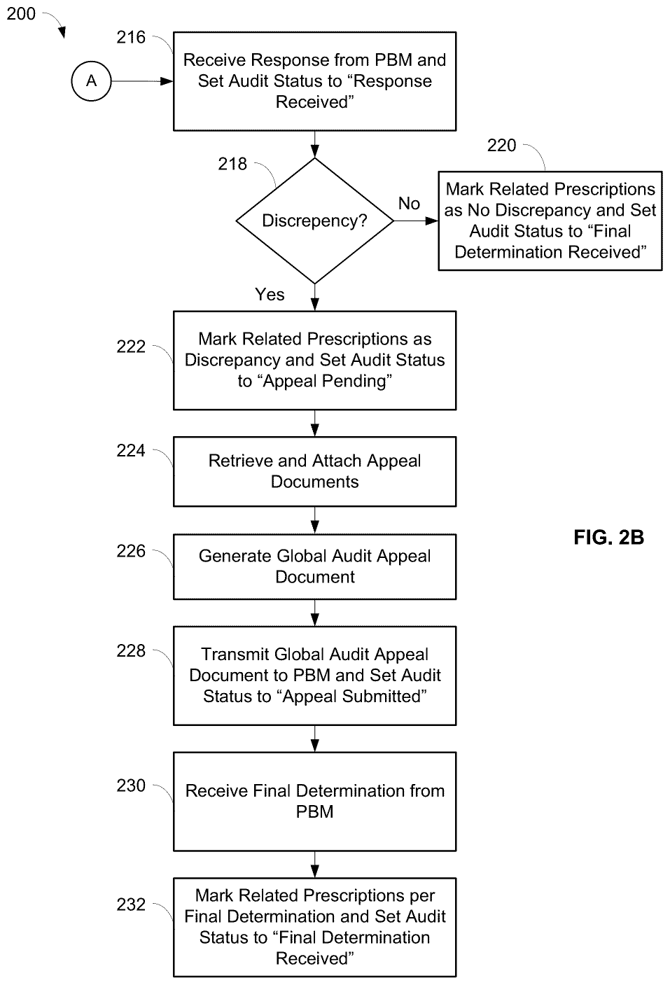

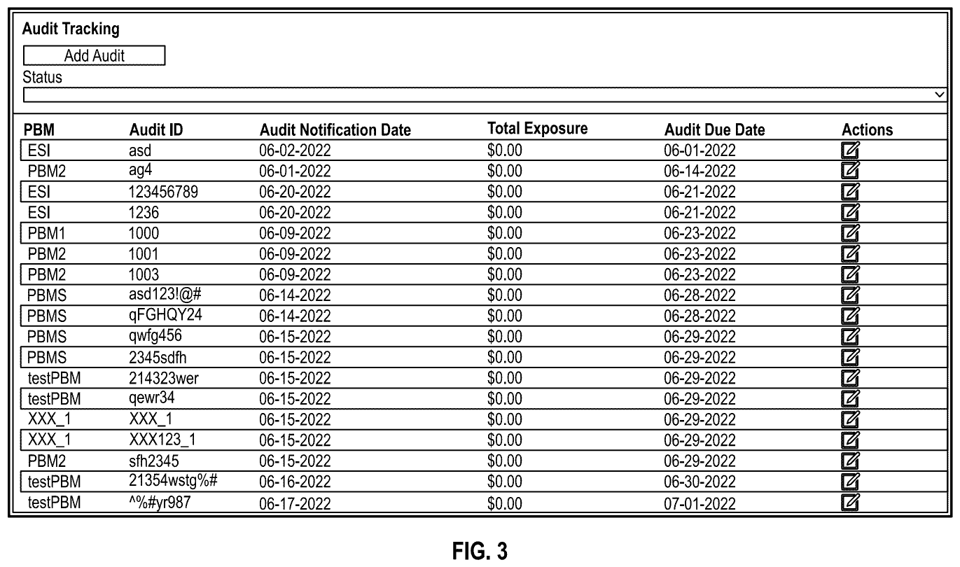

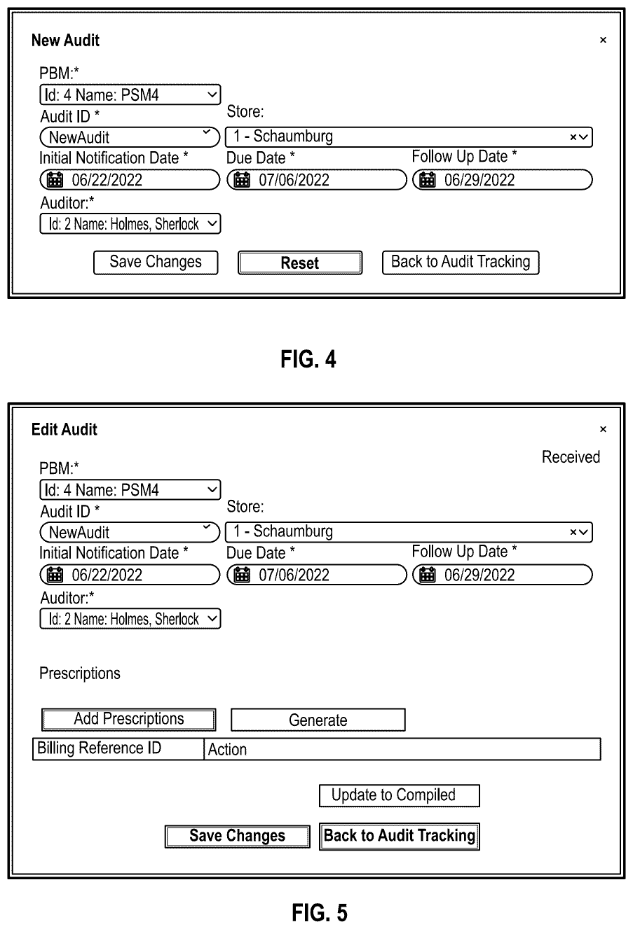

An exemplary embodiment of a process 200 related to the system 100 is shown in FIGS. 2A-2B. One or more processors and/or other processing components (e.g., analog to digital converters, encryption chips, etc.) may perform any, some, or all of the steps of the process 200. One or more other types of components (e.g., user interface 106, transmitters, receivers, buffers, drivers, discrete components, etc.) may also be utilized in conjunction with the processors and/or other processing components to perform any, some, or all of the steps of the process 200. Exemplary screenshots of software that interfaces with and implements the system 100 and the process 200 are shown in FIGS. 3-21, and are described in more detail below.

The various components of the system 100 may be communicatively coupled by a system bus, network, or other connection mechanism. A processor executing the process 200 may include a general purpose processor (e.g., a microprocessor) and/or a special purpose processor. The processor may be any suitable processing device or set of processing devices such as, but not limited to, a microprocessor, a microcontroller-based platform, an integrated circuit, one or more field programmable gate arrays (FPGAs), and/or one or more application-specific integrated circuits (ASICs).

The user interface 106 may facilitate interaction with a user of the system 100. As such, the user interface 106 may include input components such as a keyboard, a keypad, a mouse, a touch-sensitive panel, a sound speaker, or a haptic feedback system. The user interface 106 may also comprise devices that communicate with inputs or outputs, such as a short-range transceiver (RFID, Bluetooth, etc.), a telephonic interface, a cellular communication port, a router, or other types of network communication equipment. The user interface 106 may be internal to a computing device, or may be external and connected wirelessly or via connection cable, such as through a universal serial bus port. A display may be part of the user interface 106 and may include a screen, which for example, may be combined with a touch-sensitive panel. The user interface 106 may display interaction elements for a user to utilize to control the system 100 and/or the software, e.g., buttons, icons, menus, dropdown boxes, etc.

A memory for the database 108 and/or for storing instructions for the process 200 may be implemented and stored in volatile memory (e.g., RAM including non-volatile RAM, magnetic RAM, ferroelectric RAM, etc.), non-volatile memory (e.g., disk memory, FLASH memory, EPROMs, EEPROMs, memristor-based non-volatile solid-state memory, etc.), unalterable memory (e.g., EPROMs), read-only memory, and/or high-capacity storage devices (e.g., hard drives, solid state drives, etc.). In some examples, the memory may include multiple kinds of memory, particularly volatile memory and non-volatile memory. The memory may be computer readable media on which one or more sets of instructions, such as the software for operating the methods of the present disclosure can be embedded. The instructions may embody one or more of the methods or logic as described herein. As an example, the instructions can reside completely, or at least partially, within any one or more of the memory, the computer readable medium, and/or within the processor during execution of the instructions.

A request for a prescription audit may be received at the system 100, e.g., at the audit management module 102, from a pharmacy benefit manager server 150, such as at step 202 of the process 200 shown in FIG. 2A. In embodiments, the request for a prescription audit may be received at step 202 from a pharmacy benefit manager via fax or email. The request for the prescription audit received at step 202 may include an audit identifier, information related to the particular prescriptions related to the audit (e.g., order numbers, etc.), locations of the pharmacy where the prescriptions were filled, and/or other information. As examples, the pharmacy benefit manager may request a variety of documents including, but not limited to, the original image of the prescription, evidence that the patient received the medication, evidence that the prescription was processed with the correct information, evidence that the patient paid the co-pay, etc.

Audits may be tracked using a dashboard that may be displayed and/or interacted with using the user interface 106, such as the exemplary Audit Tracking dashboard shown in FIG. 3. The Audit Tracking dashboard may display all pending audits for the pharmacy, and include information such as the pharmacy benefit manager (PBM) that requested an audit, an audit identifier, an audit notification date (e.g., when an audit request was received), total monetary exposure for the pharmacy related to the audit (e.g., related to clawbacks), and/or an audit due date (e.g., when the audit needs to be completed by). The dashboard may also include the ability for a user to initiate various actions related to specific audits, and/or to add information regarding a new audit (e.g., “Add Audit” button).

An audit may have an associated status to indicate the current state of the processing and handling of the audit. The status of an audit may be displayed on the user interface 106 on various screens, such as the dashboard. Possible exemplary statuses of an audit are described in more detail below, and may include “Received”, “Compiled”, “Submitted”, “Response Received”, “Appeal Pending”, “Appeal Submitted”, and “Final Determination Received”. It is contemplated and possible that more, less, and/or other statuses of the audit may be utilized during the process and handling of the audit. In some embodiments, the status of the audit may be manually set at one or more of the various steps of the process 200, e.g., by the user. In other embodiments, the status of the audit may be automatically set at one or more of the various steps of the process 200, e.g., in response to an action by the user and/or the receipt of particular data.

The status of the audit may be set to “Received” at step 202 to indicate that an audit request has been received from a PBM and that a new audit is to be created. After receiving an audit request at step 202, a user may create a new audit (e.g., by clicking the “Add Audit” button on the dashboard) and enter audit creation information to be received by the audit management module 102 at step 204. An exemplary New Audit form for creating a new audit is shown in FIG.

4. The audit creation information entered at step 204 may include information from the audit request, such as the PBM's name, audit identifier, audit notification date (e.g., initial notification date), and audit due date. The audit creation information may also include a location of a pharmacy related to the audit, a follow-up date, and/or a name of the person responsible for handling the audit (e.g., “Auditor”). Some or all of the audit creation information may be manually entered by a user at step 204. In embodiments, some or all of the audit creation information may be automatically captured and entered at step 204 by the audit management module 102, such as when the audit management module 102 processes the audit request received at step 202.

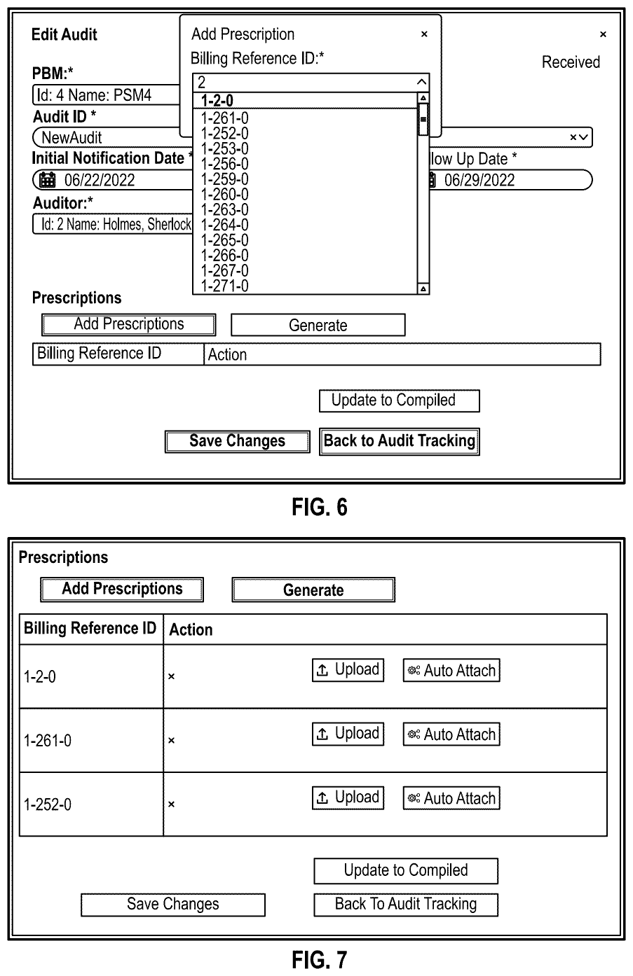

As shown in FIG. 5, an exemplary Edit Audit form may also be used at step 204 to allow entry of information related to each prescription associated with the audit request. The information related to each prescription may include a billing reference identifier and/or prescription documentation, for example. A user may click on the “Add Prescriptions” button which may bring up an Add Prescription form, as shown in the exemplary screenshot of FIG. 6. The Add Prescription form may include a drop down of billing reference identifiers that can be selected by a user to add to the new audit being created. In embodiments, the billing reference identifiers shown in the drop down of the Add Prescription form may be filtered by a particular pharmacy location related to the audit, in order to avoid selecting prescriptions from other pharmacy locations that are not related to the audit. A user may also manually enter a billing reference identifier in the Add Prescription form. An exemplary list of prescriptions that have been added to an audit is shown in FIG. 7.

In addition to the billing reference identifiers, documentation may be added to a prescription that is related to an audit. Such documentation may include, for example, proof of request of the prescription (e.g., an online order), a hard copy of the prescription, a “processed screen” (e.g., printout of the audit screen), a shipping ticket, an auto-refill attestation, and/or other documentation. At step 206, it may be determined whether prescription-related documents are available to be automatically attached to the audit being created. If prescription-related documents can be automatically attached at step 206, then the process 200 may continue to step 210. At step 210, the audit management module 102 may search the database 108 to find and retrieve relevant documents to be attached to the audit. The audit management module 102 may, for example, query the database 108 using the billing reference identifier to retrieve the relevant documents. In some embodiments, a user may click on the “Auto Attach” button for a prescription (e.g., as shown in FIG. 7) to perform the automatic attachment of the prescription-related documents. In other embodiments, the audit management module 102 may perform the automatic attachment of the prescription-related documents, e.g., when a prescription has been added to the audit at step 204. The prescription-related documents may be in any suitable format, such as Portable Document Format (PDF), images (JPEG, PNG, GIF, BMP, TIFF, etc.), plain text, Microsoft Word (DOC, DOCX, etc.), HyperText Markup Language (HTML), Extensible Markup Language (XML), Encapsulated PostScript (EPS), Microsoft Excel (XLS, XLSX, etc.), Comma Separated Values (CSV), etc.

If prescription-related documents can not be automatically attached at step 206, then the process 200 may continue to step 208. At step 208, the prescription-related documents may be manually attached by a user. For example, the user may search the database 108 and/or drag and drop the relevant prescription-related document files to be attached to the audit at step 208. In embodiments, both the automatic attachment of documents at step 210 and the manual attachment of documents at step 208 may occur. This could happen, for example, if not all of the relevant prescription-related documents can be automatically found and attached at step 210.

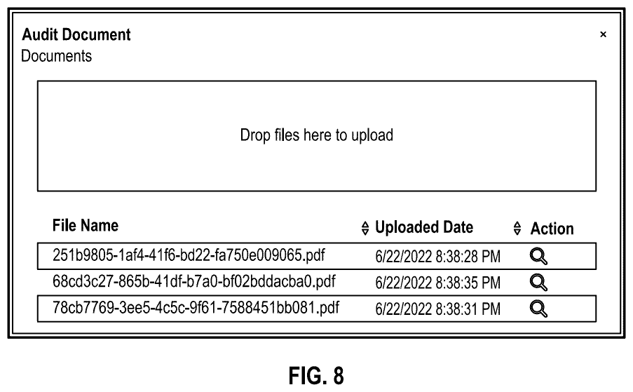

An exemplary screenshot listing the prescription-related documents that have been attached to an audit is shown in FIG. 8. The listing of the prescription-related documents as shown in FIG. 8 may occur following the automatic attachment of documents at step 210 and/or the manual attachment of documents at step 208. As seen in FIG. 8, each of the prescription-related documents may be attached such that they have a unique filename and timestamp of when they were uploaded. In addition, a user may view the prescription-related documents, e.g., by opening a PDF, to verify that they are the correct documents for the audit. A user may also delete prescription-related documents that were previously attached, if necessary.

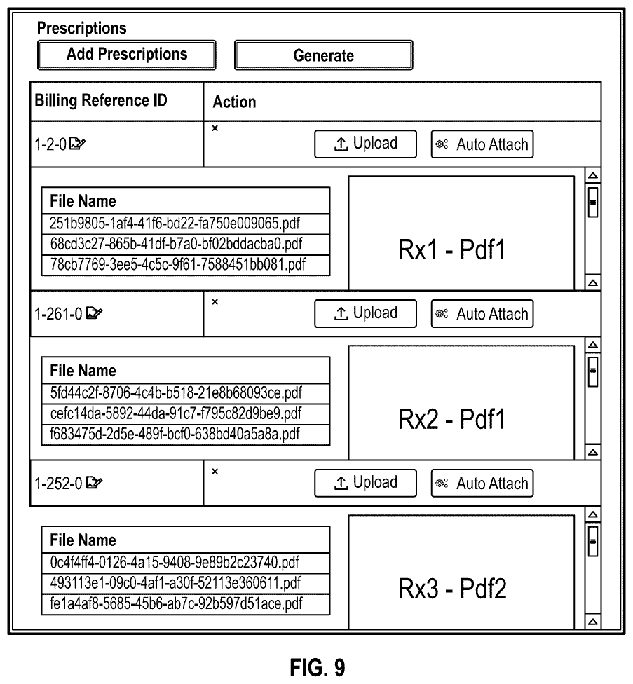

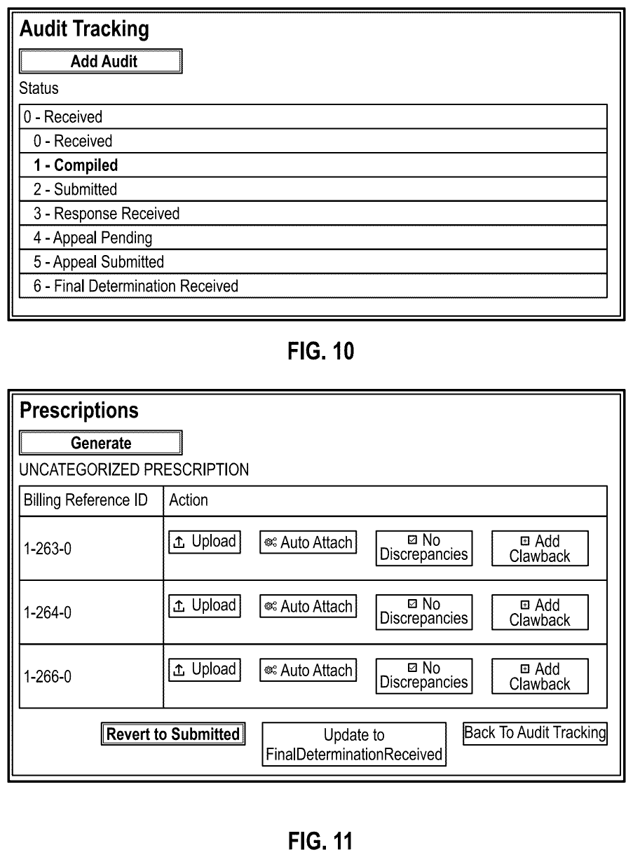

Following step 208 or step 210, the process 200 may continue to step 212. At step 212, a global audit response document may be generated by the report generation module 104. In embodiments, the global audit response document generated at step 212 may include all of the prescription-related documents that were previously attached at steps 208 and/or 210. The global audit response document may be a PDF or other suitable file type. As shown in the exemplary screenshot of FIG. 9, the global audit response document may include each prescription related to the audit (e.g., listed by billing reference identifier), as well as the documents related to each of the prescriptions. The global audit response document may be viewed by a user to ensure that it is complete and accurate. The status of the audit at step 212 may be set to “Compiled” (e.g., using the dropdown box shown in FIG. 10) to indicate that the relevant prescriptions and related documentation have been gathered and attached to the audit.

Following step 212, the process 200 may continue to step 214. At step 214, the global audit response document generated at step 212 may be transmitted to the pharmacy benefit manager server 150 from the system 100, e.g., from the report generation module 104 via the audit management module 102. In embodiments, the global audit response document generated at step 212 may be transmitted at step 214 to the pharmacy benefit manager via fax or email. In addition, the status of the audit at step 214 may be set to “Submitted” to indicate that the pharmacy has responded to the audit request from the pharmacy benefit manager by sending the global audit response document.

As denoted by the node A shown in FIGS. 2A-2B, the process 200 continues after step 214 on FIG. 2A to step 216 on FIG. 2B. At step 216, a response may be received at the audit management module 102 from the pharmacy benefit manager server 150. The PBM response may have been received after the pharmacy benefit manager has reviewed the global audit response document that was transmitted at step 214. The PBM response received at step 216 may include a listing of (1) prescriptions where no discrepancies were found and (2) prescriptions where discrepancies were found. For example, the pharmacy benefit manager may determine that a filled prescription had a discrepancy when the directions were inaccurate or there was insufficient documentation. In embodiments, the PBM response may be received at step 216 from a pharmacy benefit manager via fax or email.

The prescriptions where discrepancies were found may also include a clawback value that indicates an amount that the pharmacy benefit manager believes should be paid to them for that particular prescription, e.g., to compensate for an incorrect initial payment. The PBM response received at step 216 may also include a total of all of the clawback values for the prescriptions where discrepancies were found. In addition, the status of the audit at step 216 may be set to “Response Received” to indicate that the pharmacy has received an initial response to the global audit response document from the pharmacy benefit manager.

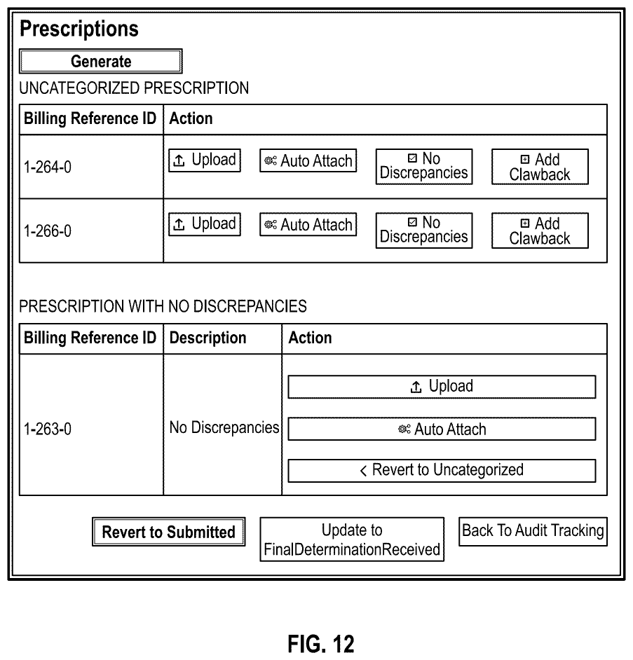

Following step 216, the process 200 may continue to step 218. At step 218, it may be determined whether the PBM response includes any prescriptions where discrepancies were found. If it is determined at step 218 that the PBM response does not include any prescriptions with discrepancies, then the process 200 may continue to step 220. At step 220, the particular prescriptions that do not have discrepancies may be marked accordingly in the database 108. As seen in the exemplary list of prescriptions shown in FIG. 11, a user may click the “No Discrepancies” button for each particular prescription that does not have a discrepancy. When a particular prescription is marked as not having a discrepancy, then it may be marked accordingly and moved to the “Prescription with No Discrepancies” portion of the listing, as shown in the exemplary screenshot of FIG. 12. In addition, the status of the audit at step 220 may be set to “Final Determination Received” to indicate that the audit has been completed, and the process 200 may be complete. In this scenario, since there are no prescriptions that have discrepancies, then no clawback payments need to be made to the PBM from the pharmacy.

However, if it is determined at step 218 that the PBM response received at step 216 includes one or more prescriptions with discrepancies, then the process 200 may continue to step 222. At step 222, the particular prescriptions that have discrepancies may be marked accordingly in the database 108. For example, a user may click the “Add Clawback” button, as shown in FIGS. 11-12, to denote that a particular prescription has a discrepancy in the PBM response. The particular prescriptions that do not have discrepancies may also be marked accordingly in the database 108 at step 222. In embodiments, the marking of prescriptions that do not have discrepancies or do have discrepancies (e.g., at step 220 and step 222, respectively) may automatically occur, such as when the audit management module 102 processes the PBM response received at step 216.

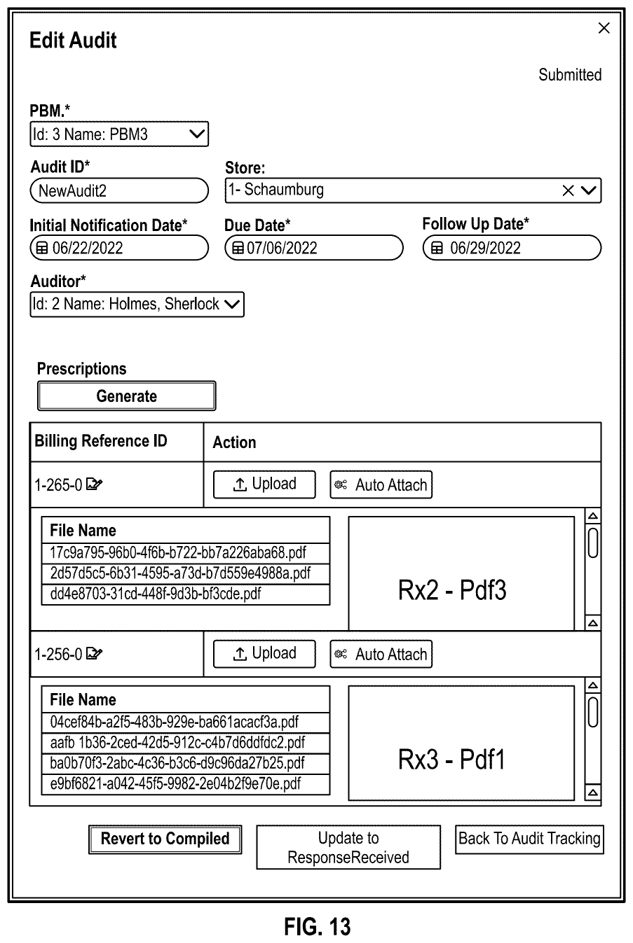

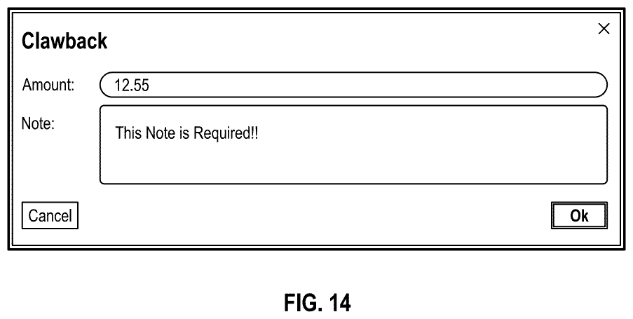

An appeal of the prescriptions with discrepancies may also be created at step 222. Such an appeal may be created to contest with the PBM the particular prescriptions with discrepancies included in the PBM response received at step 216. As shown in the exemplary Edit Audit form shown in FIG. 13, certain prescriptions that had discrepancies may be appealed. Prescriptions with discrepancies may be routinely appealed based on the reasons for clawback provided by the pharmacy benefit manager. For prescriptions that have been marked as needing a clawback (e.g., at step 222), the clawback value and a note explaining reasons the clawback is being appealed may be entered, such as shown in the exemplary Clawback form shown in FIG. 14.

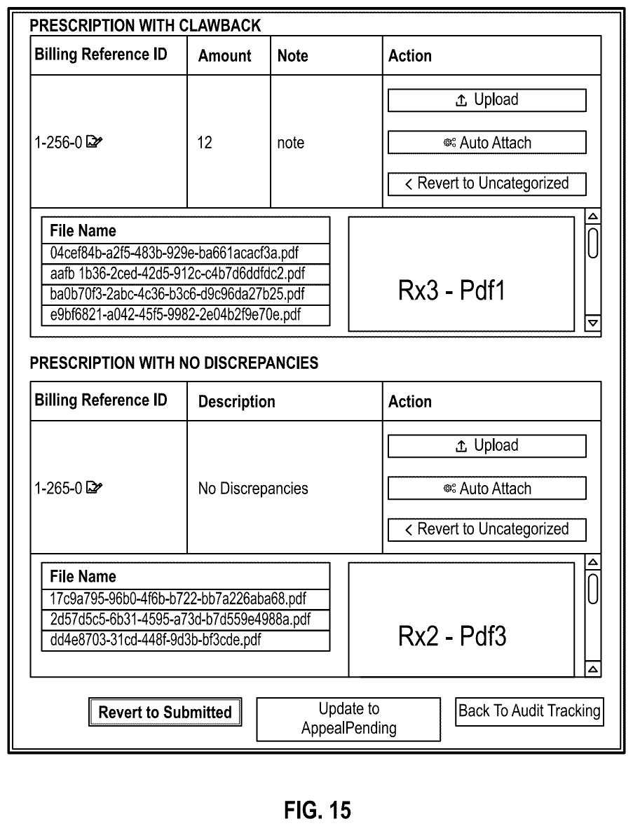

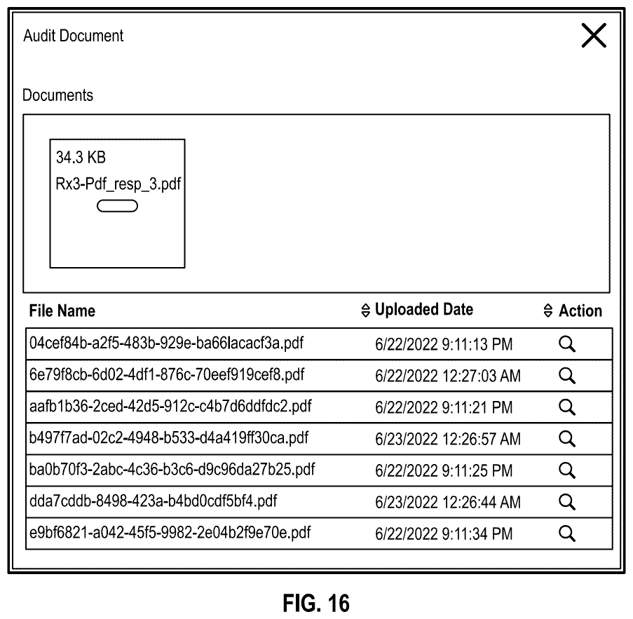

As shown in FIG. 15, prescriptions with discrepancies in the PBM response may be listed as “Prescription with Clawback” while prescriptions with no discrepancies in the response from the PBM may be listed as “Prescription with No Discrepancies”. For purposes of the appeal for prescriptions with discrepancies, further documentation may be attached related to the appeal. Such documentation may include, for example, an attestation from the patient and/or prescriber, or an explanation of the facts related to the filling of the prescription (e.g., to help the auditor understand the reasoning for the appeal). The documentation for the appeal of prescriptions with discrepancies may be manually attached by a user and/or automatically attached, e.g., at step 224 and as shown in the exemplary screenshot of FIG. 16.

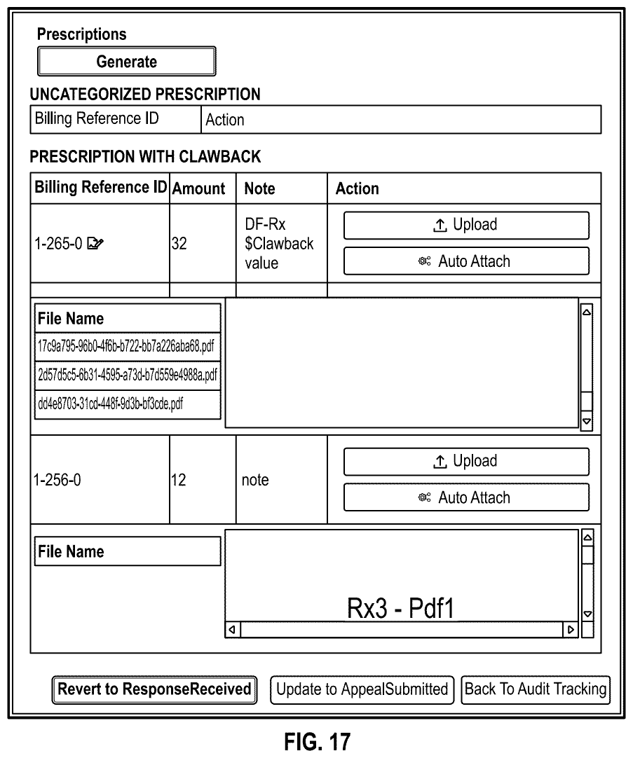

Following step 224, a global audit appeal document may be generated at step 226 by the report generation module 104. The global audit appeal document may include, for example, the prescription-related documents that were previously part of the global audit response document (e.g., generated at step 212) and/or additional documents for the appeal that were attached at step 224. The global audit appeal document may also include the note explaining reasons the clawback is being appealed. The global audit appeal document may be a PDF or other suitable file type. As shown in the exemplary screenshot of FIG. 17, the global audit appeal document may include each prescription related to the appeal (e.g., listed by billing reference identifier), as well as the documents related to the appeal. The global audit appeal document may be viewed by a user to ensure that it is complete and accurate.

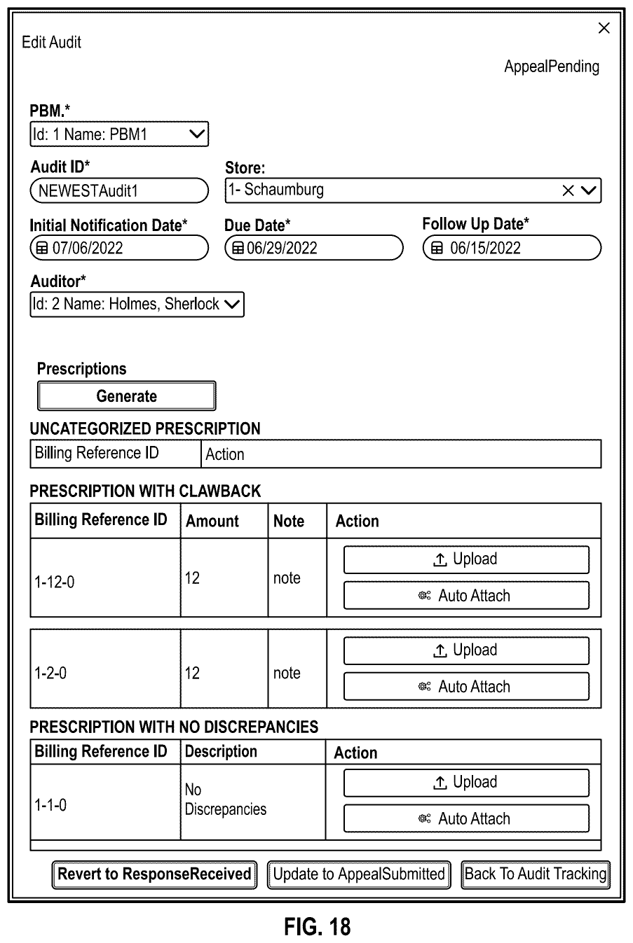

Following step 226, the process 200 may continue to step 228. At step 228, the global audit appeal document generated at step 226 may be transmitted to the pharmacy benefit manager server 150 from the system 100, e.g., from the report generation module 104 via the audit management module 102. In embodiments, the global audit appeal document generated at step 226 may be transmitted at step 228 to a pharmacy benefit manager via fax or email. In addition, the status of the audit at step 228 may be set to “Appeal Submitted” to indicate that the pharmacy has appealed the response from the PBM, e.g., as shown in the exemplary screenshot of FIG. 18.

At step 230, a final determination of the appeal may be received at the audit management module 102 from the pharmacy benefit manager server 150. The final determination may have been received after the pharmacy benefit manager has reviewed the global audit appeal document that was transmitted at step 228. The final determination received at step 230 may include whether an appealed prescription that had discrepancies has been changed to no discrepancies, whether a discrepancy status has been maintained by the PBM for the appealed prescription (e.g., to still require payment of the clawback value), and/or whether the clawback value for an appeal prescription has been changed by the PBM. In embodiments, the final determination of the appeal may be received at step 230 from a pharmacy benefit manager via fax or email.

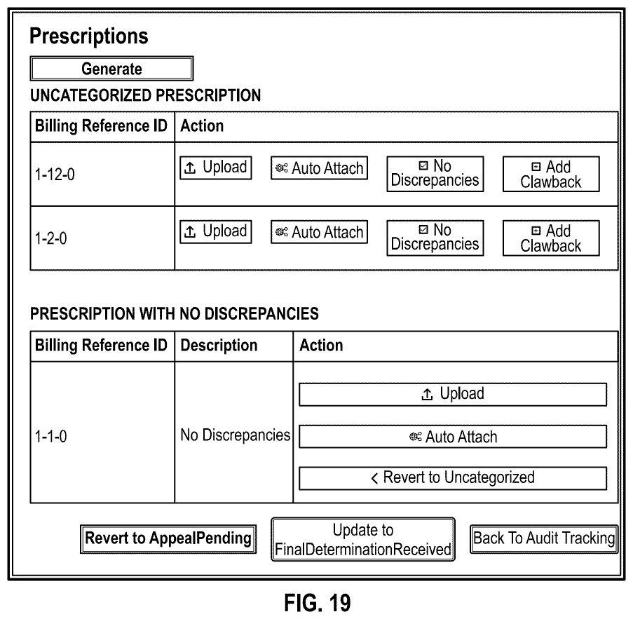

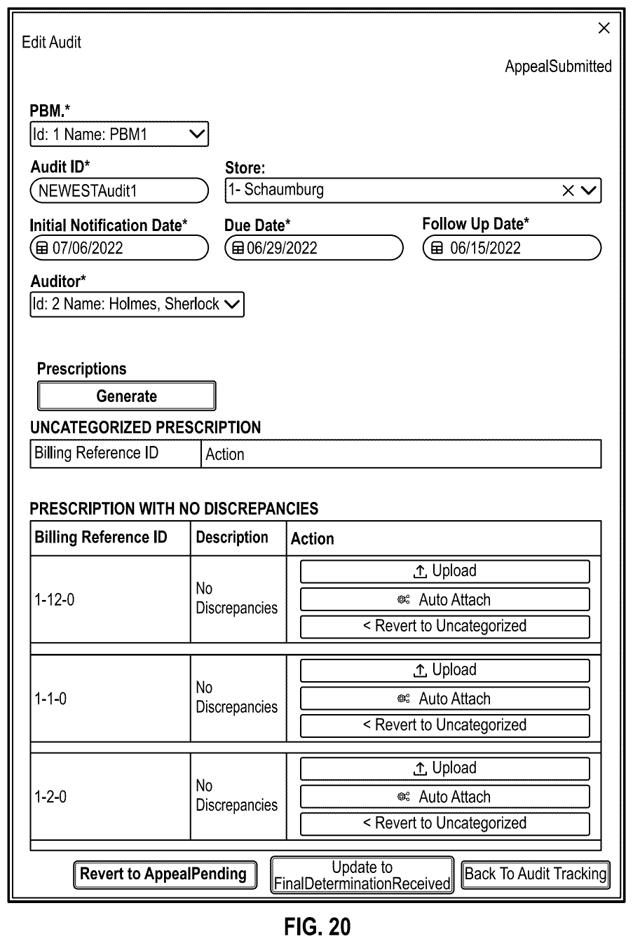

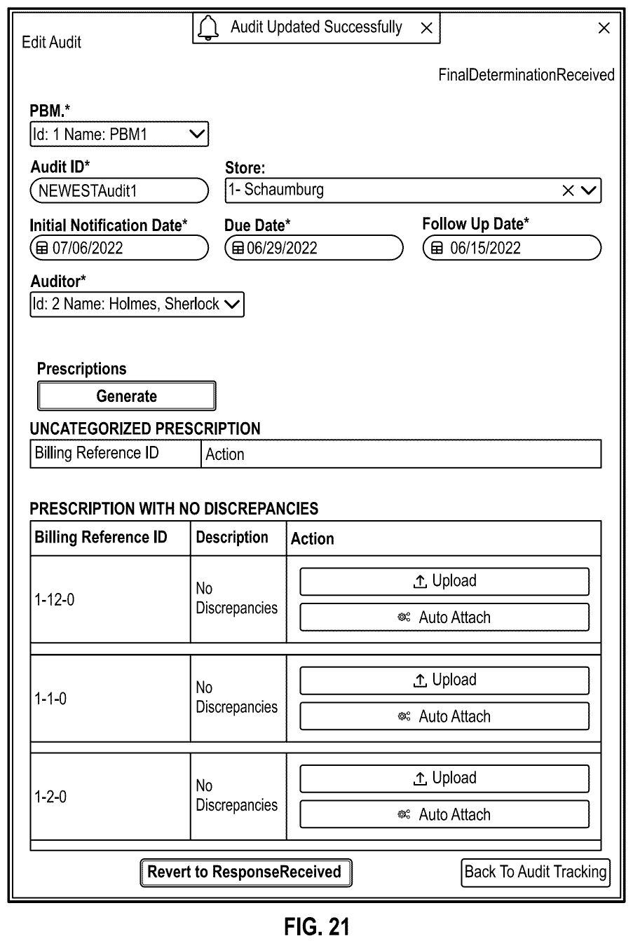

As shown in the exemplary screenshots of FIGS. 19-20 and at step 232, a user may change a prescription that had discrepancies in the initial PBM response to be marked as having no discrepancies, e.g., per the final determination received from the PBM. In embodiments, such marking of prescriptions may automatically occur, such as when the audit management module 102 processes the final determination received at step 230. In addition, the status of the audit at step 232 may be set to “Final Determination Received” to indicate that the audit has been completed, e.g., as shown in the exemplary screenshot of FIG. 21, and the process 200 may be complete.

In embodiments, the prescriptions related to an audit and/or appeal may be utilized and processed by an artificial intelligence model to determine certain traits and characteristics of prescriptions that may be audited and/or have discrepancies. For example, the artificial intelligence model may determine that prescriptions that are audited may be related to certain medications, types of medications, prescribers, patients, pharmacy locations, etc. The artificial intelligence model may utilize this information to assign audit risk scores and/or provide potential audit alerts when processing future prescriptions.

The description herein describes, illustrates and exemplifies one or more particular embodiments of the invention in accordance with its principles. This description is not provided to limit the invention to the embodiments described herein, but rather to explain and teach the principles of the invention in such a way to enable one of ordinary skill in the art to understand these principles and, with that understanding, be able to apply them to practice not only the embodiments described herein, but also other embodiments that may come to mind in accordance with these principles. The scope of the invention is intended to cover all such embodiments that may fall within the scope of the appended claims, either literally or under the doctrine of equivalents.

It should be noted that in the description and drawings, like or substantially similar elements may be labeled with the same reference numerals. However, sometimes these elements may be labeled with differing numbers, such as, for example, in cases where such labeling facilitates a more clear description. Additionally, the drawings set forth herein are not necessarily drawn to scale, and in some instances proportions may have been exaggerated to more clearly depict certain features. Such labeling and drawing practices do not necessarily implicate an underlying substantive purpose. As stated above, the specification is intended to be taken as a whole and interpreted in accordance with the principles of the invention as taught herein and understood to one of ordinary skill in the art.

Any process descriptions or blocks in figures should be understood as representing modules, segments, or portions of code which include one or more executable instructions for implementing specific logical functions or steps in the process, and alternate implementations are included within the scope of the embodiments of the invention in which functions may be executed out of order from that shown or discussed, including substantially concurrently or in reverse order, depending on the functionality involved, as would be understood by those having ordinary skill in the art.

This disclosure is intended to explain how to fashion and use various embodiments in accordance with the technology rather than to limit the true, intended, and fair scope and spirit thereof. The foregoing description is not intended to be exhaustive or to be limited to the precise forms disclosed. Modifications or variations are possible in light of the above teachings. The embodiment(s) were chosen and described to provide the best illustration of the principle of the described technology and its practical application, and to enable one of ordinary skill in the art to utilize the technology in various embodiments and with various modifications as are suited to the particular use contemplated. All such modifications and variations are within the scope of the embodiments as determined by the appended claims, as may be amended during the pendency of this application for patent, and all equivalents thereof, when interpreted in accordance with the breadth to which they are fairly, legally and equitably entitled.

1. A method for processing an audit of at least one medication prescription, comprising:
receiving, at a processor, an audit request comprising an indication of the at least one medication prescription being audited;
identifying, using the processor, one or more documents in a database in communication with the processor, wherein the one or more documents are associated with the at least one medication prescription being audited;
retrieving, from the database to the processor, the one or more documents; and
generating, using the processor, an audit document related to the at least one medication prescription being audited, wherein the audit document comprises the one or more documents and data associated with the at least one medication prescription being audited.

2. The method of claim 1, further comprising receiving, from a user interface in communication with the processor, user input related to one or more of: the one or more documents or the data associated with the at least one medication prescription being audited.

3. The method of claim 2, wherein the user input comprises an indication of the one or more documents associated with the at least one medication prescription being audited.

4. The method of claim 1, wherein generating the audit document comprises processing, using the processor, the audit request to generate the data associated with the at least one medication prescription being audited.

5. The method of claim 1, wherein identifying the one or more documents comprises querying the database, using the processor, with a reference identifier associated with the at least one medication prescription being audited to identify the one or more documents.

6. The method of claim 5, wherein the audit request further comprises the reference identifier associated with the at least one medication prescription being audited.

7. The method of claim 1, further comprising receiving, from a user interface in communication with the processor, an indication from a user verifying an accuracy of the audit document.

8. The method of claim 1, further comprising transmitting the audit document from the processor to a pharmacy benefit manager server.

9. The method of claim 1, further comprising displaying, on a user interface in communication with the processor, a current status of the audit of the at least one medication prescription being audited.

10. The method of claim 1, further comprising:
receiving, at the processor, an audit response comprising an indication of a subset of the at least one medication prescription being audited that has a discrepancy; and
updating the database, using the processor, to denote that the subset of the at least one medication prescription being audited has the discrepancy.

11. The method of claim 10, wherein the audit response further comprises a clawback value associated with the subset of the at least one medication prescription being audited that has the discrepancy.

12. The method of claim 10, wherein the discrepancy comprises an indication of insufficient documentation related to the subset of at least one medication prescription being audited that has the discrepancy.

13. The method of claim 10, further comprising:
identifying, using the processor, one or more prescription appeal documents in the database, wherein the one or more prescription appeal documents are associated with the subset of the at least one medication prescription being audited that has the discrepancy;
retrieving, from the database to the processor, the one or more prescription appeal documents; and
generating, using the processor, an appeal document related to the subset of the at least one medication prescription being audited that has the discrepancy, wherein the appeal document comprises the one or more prescription appeal documents and appeal data associated with the subset of the at least one medication prescription being audited that has the discrepancy.

14. The method of claim 13, further comprising transmitting the appeal document from the processor to a pharmacy benefit manager server.

15. The method of claim 13, further comprising:
receiving, at the processor, an appeal response comprising an indication of a final determination related to the subset of the at least one medication prescription being audited that has the discrepancy; and
updating the database, using the processor, to denote that the subset of the at least one medication prescription being audited that has the discrepancy has had a final determination.

16. The method of claim 1, further comprising processing, using an artificial intelligence algorithm executing on the processor, data associated with the at least one medication prescription to determine one or more characteristics of the at least one medication prescription that is likely to be audited.

17. The method of claim 16, further comprising generating, using the artificial intelligence algorithm executing on the processor, a risk score associated with the at least one medication prescription, wherein the risk score relates to a likelihood of the at least one medication prescription being audited.

18. A system for processing an audit of at least one medication prescription, comprising:
a user interface;
a database comprising one or more documents associated with the at least one medication prescription being audited; and
a processor in communication with the user interface and the database, the processor configured to:
receive an audit request comprising an indication of the at least one medication prescription being audited;
identify in the database the one or more documents associated with the at least one medication prescription being audited;
retrieve the one or more documents from the database; and
generate an audit document related to the at least one medication prescription being audited, wherein the audit document comprises the one or more documents and data associated with the at least one medication prescription being audited.

19. The system of claim 18, wherein the processor is configured to generate the audit document by processing the audit request to generate the data associated with the at least one medication prescription being audited.

20. The system of claim 18, wherein the processor is configured to identify the one or more documents by querying the database with a reference identifier associated with the at least one medication prescription being audited to identify the one or more documents.
